(19) United States
(12) Patent Application Publication
(10) Pub. No.: US 2021/0153977 A1
(43) Pub. Date: May 27, 2021

(54) DENTAL ALIGNER AND METHOD FOR PRODUCTION

(71) Applicant: International Association For Medical and Dental Research Limited, Kowloon (HK)

(72) Inventor: Cecilia YOUNG, Kowloon (HK)

(73) Assignee: International Association For Medical and Dental Research Limited, Kowloon (HK)

(21) Appl. No.: 17/100,733

(22) Filed: Nov. 20, 2020

Related U.S. Application Data
(60) Provisional application No. 62/938,530, filed on Nov. 21, 2019.

(51) Int. Cl. A61C 7/00; A61C 7/08; A61C 7/36; B33Y 50/00; B33Y 10/00; B33Y 80/00

(52) U.S. Cl. CPC A61C 7/002; A61C 7/08; A61C 7/36; A61B 6/14; B33Y 10/00; B33Y 80/00; B33Y 50/00

(57) ABSTRACT

A method for producing dental aligners includes scanning a patient's teeth to produce a 3D printable file, adjusting the 3D printable file according to a treatment plan, printing a dental aligner mold based on the adjusted 3D printable file and producing dental aligners from the dental aligner mold. The method may be implemented as a computer software program and may include additional features such as setting guidelines and A.I. suggestions for optimal movements.

### BACKGROUND

### Field of the Invention

The present invention relates generally to dental aligners, and more specifically, to a method for producing dental aligners for all directions and optimizing adjustments based on a particular treatment plan.

### Description of Related Art

Methods for producing dental aligners are known in the art. However due to the limitation of the material, the strength and the force provided by the aligners is weak. The increase of the thickness will not overcome the problem because the aligner could not be seated if it is too hard or thick.

Additionally, most methods are not user friendly, particularly those that require a dentist or orthodontist to use specific programs. Dentists and orthodontists just use it to achieve the goal. There is a great need for a method that allows dentists and orthodontists to make quick and easy adjustments to a dental aligner according to a particular patient's treatment plan.

Accordingly, although great strides have been made in the area of methods for producing dental aligners, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIGS. 1-16 depict various embodiments of a dental aligner;

FIG. 17 is a flowchart of a method for producing dental aligners in accordance with a preferred embodiment of the present application;

FIG. 18 is a top view of a lower portion of a dental aligner in accordance with a preferred embodiment of the present application; and

FIG. 19 is a top view of an upper portion of the dental aligner.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional methods for producing dental aligners. Specifically, the method allows for dentists and orthodontists to optimize adjustments to a dental aligner according to a specific treatment plan. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

The present invention is that the varies thickness and different material (e.g. soft and hard thermoplastic) at different area of the aligners to combat the above problem. The part for simply connection or light force will be thinner than the area that larger force, more abrasion, more likely be distorted or fatigue or addition of the component is needed.

The component could be completely inside or pierce out of the aligner, embedded in the outer and inner shell of the material. The component could be a hook, a button, a screw for closing (traction) or opening (expansion), a spring, a rod or a plate in any shape suit the area of metal, alloy, acrylic, thermoplastic, ceramic, composite resin, polymeric materials, polycarbonate and polyurethane.

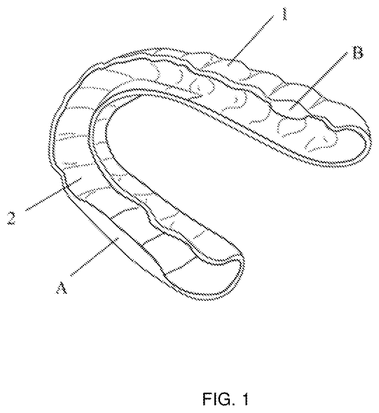

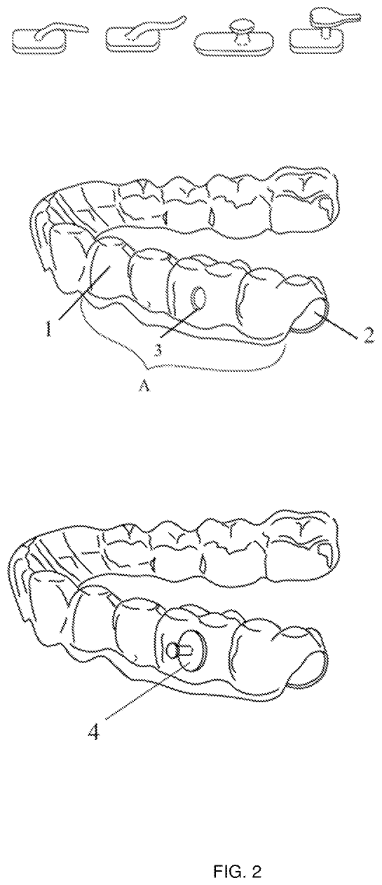

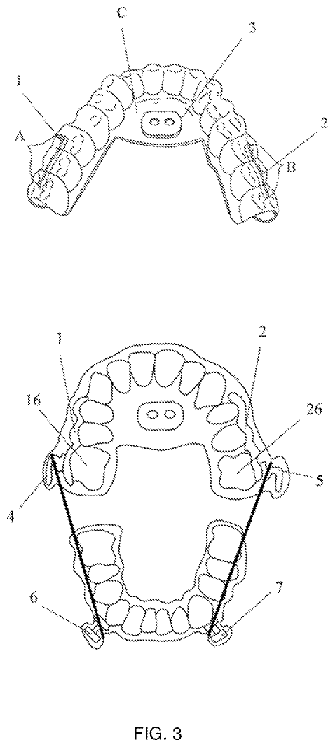

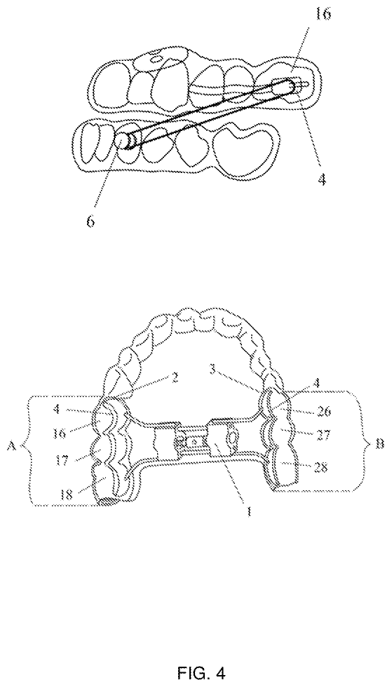

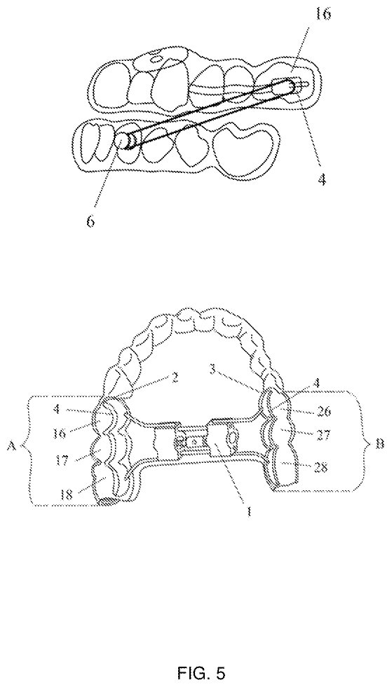

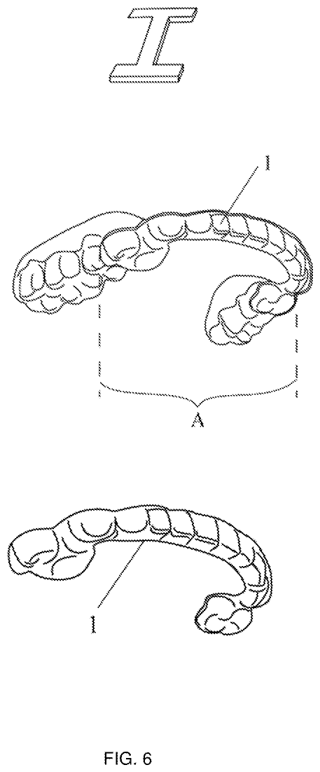

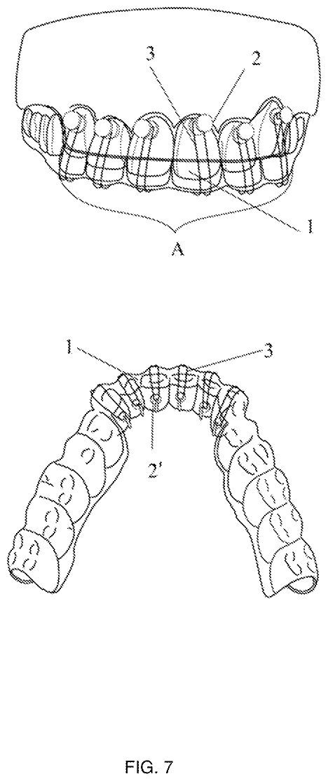

For example, FIGS. 1-7 depicts the aligner with different thickness at different area. The components could be pre-filmed with the thermoplastic material or other materials.

The component could be a light emitting source e.g. LED light or optical guide plane for the Low Level Light therapy resulted in photobiomodulation of low level light therapy, i.e. the modification of rate of the cell activity thus to make orthodontic treatment faster, promote the healing of mucosa and bone after the orthognathic surgery, palatal expansion, ulceration etc.

The process could also be used to provide the surgical guide for implant placing for anchorage/addition of implant supported prosthesis etc. Guide for microperforation could be fabricated to increase the rate of the orthodontic treatment. These are due to the cooperation of the x-ray and cone beam computerized tomography CBCT.

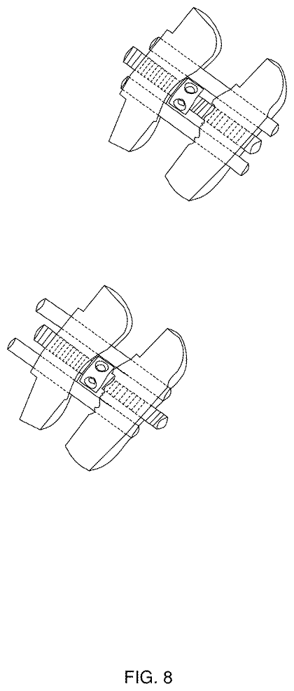

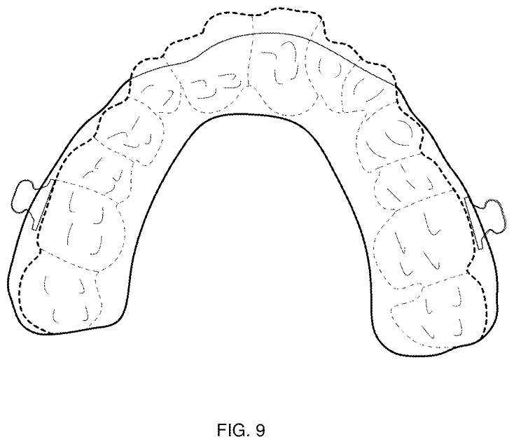

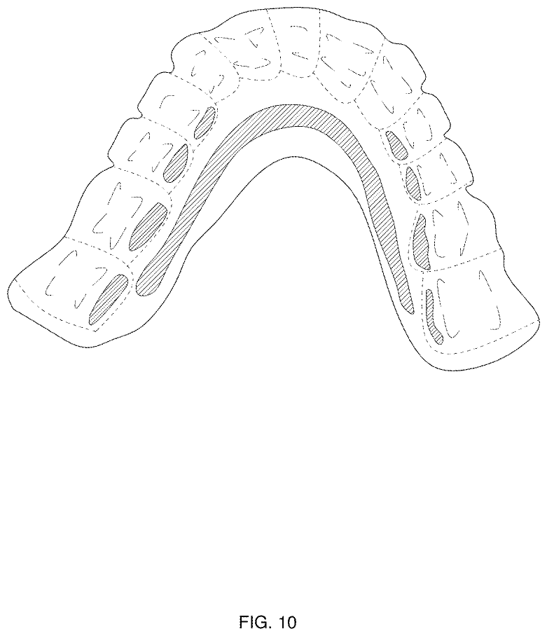

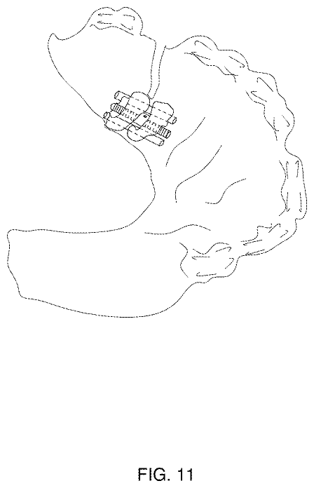

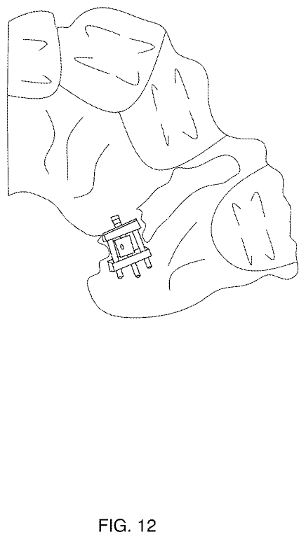

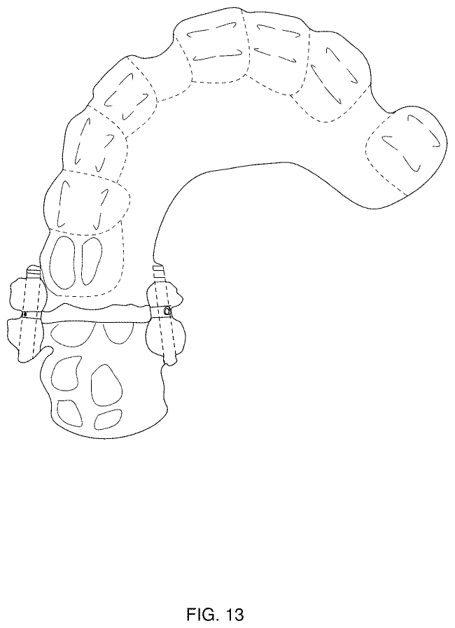

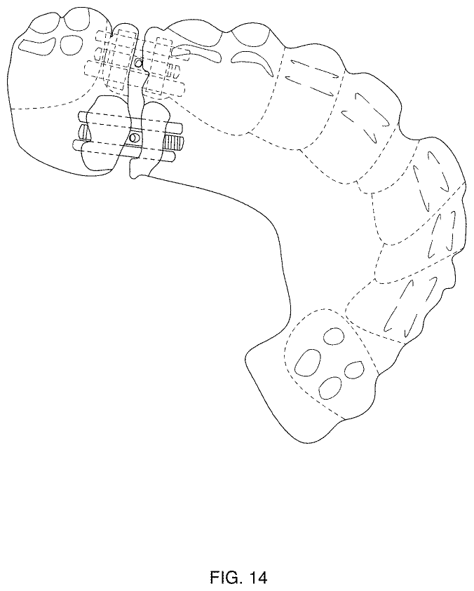

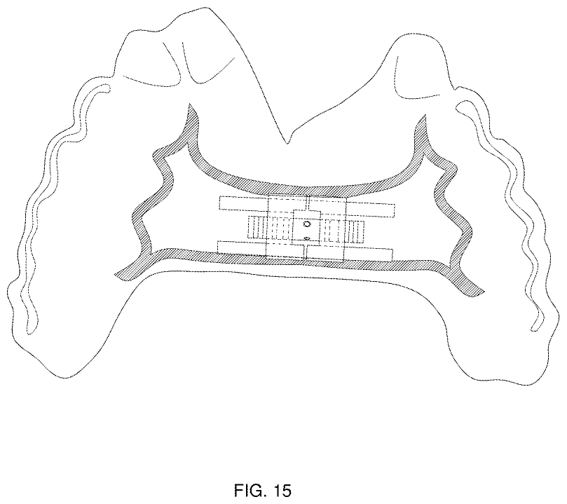

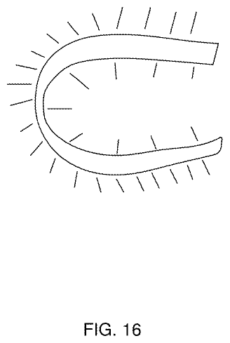

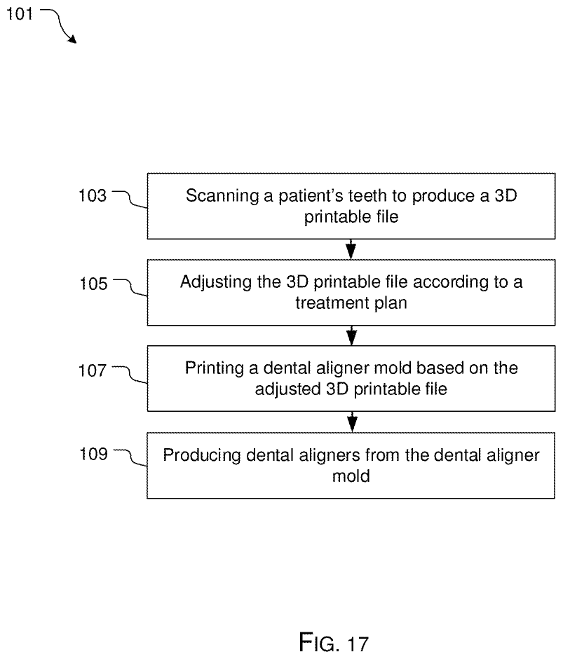

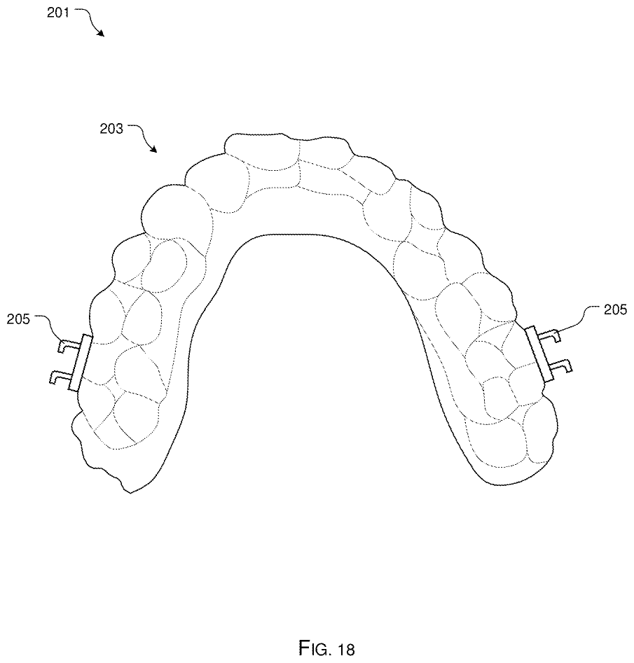

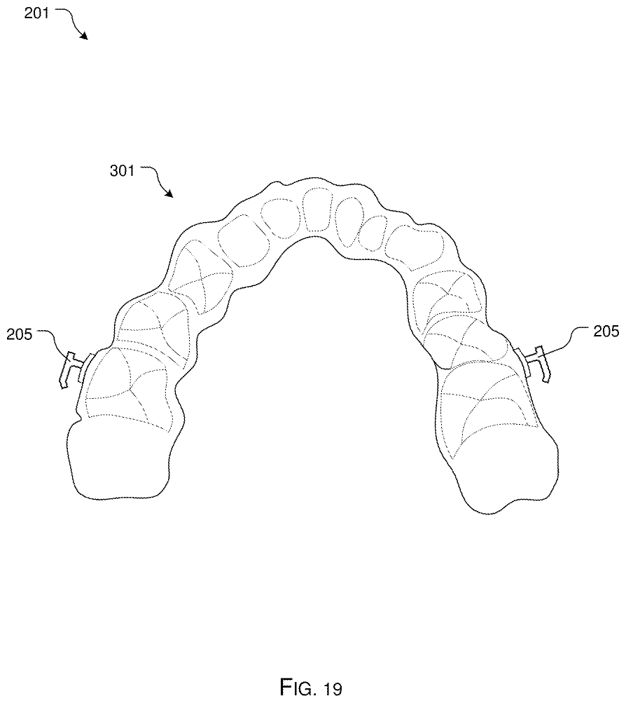

FIGS. 8 and 9 depict button incorporated in the aligner, that part is thicker and stronger with the outer and inner surface, it could be made with the vacuum form of sheets +/−3D printing. It could be any component mentioned above or e.g. of prefilmed components.

Referring now to the drawings wherein like reference characters identify corresponding or similar elements throughout the several views, FIG. 1 depicts a flowchart of a method for producing dental aligners in accordance with a preferred embodiment of the present application. It will be appreciated that method 101 overcomes one or more of the above-listed problems commonly associated with conventional methods for producing dental aligners.

In the contemplated embodiment, method 101 includes the steps of scanning a patient's teeth to produce a 3D printable file in step 103, adjusting the 3D printable file according to a treatment plan in step 105, printing a dental aligner mold based on the adjusted 3D printable file in step 107 and producing dental aligners from the dental aligner mold in step 109.

Method 101 may be implemented on a computer device as a software program. Method 101 is intended to be more user friendly and save time for dentists and orthodontists allowing for quick and accurate adjustments.

The printed models for the tooth movement may be separated into many steps from beginning to end before a final planned position is determined. Method 101 allows for the movement of a patient's teeth virtually in all directions.

The patient's teeth may be scanned using CBCT or a similar method of computer scanning of bone and teeth. This allows the combination of the bone with the teeth such that the tooth root will not come out of the bone causing pulpal death.

Method 101 may allow for A.I. or smart suggestions to an operator for maximum intercuspation. Method 101 may also include a means for determining and identifying the shortest path for a movement to achieve a desired result.

Method 101 may further include the ability for the operator to set different guidelines or defining or identifying a gumline. Method 101 may also duplicate a part of tooth for fabrication such as to strengthen an area. Method 101 may also include a reminder to add a soft layer or extra layer in certain areas as needed. This may include a relief area for a metal alloy or acrylic component to be added or an indentation on a relief area for holding the component for ease in fabrication.

The dental aligner of the present application combats limitations of conventional dental aligner systems. The dental aligner 201 includes a lower jaw portion 203 and an upper jaw portion 301. Conventional dental aligners have a uniform thickness in each aligner to balance the retention, dislodgement, force and wearing. However, the present application teaches a dental aligner with a variable thickness to improve the function of the aligners. A thinner layer for teeth that do not need to be moved or need light force or less retention. A thicker layer to strengthen area that need harness to increase the strength or force for the movement or for the anchorage area.

The dental aligners may include various auxiliaries 205 such as brackets, hooks, screws, coils, springs, trans palatal bars, strengtheners, acrylic pads and the like to improve the force for the desired movement. These auxiliaries 205 are interlocked or embedded with the dental aligners forming a unitary structure. The need to bond to teeth for most cases is decreased thus decreasing the time required.

The dental aligners may relieve the area for strengthener on the models to prevent pressing the gum or the teeth. This may be done by adding a composite spacer or other material on the casts.

The anchorage area may include a soft base to decrease the discomfort at the pressed area. It may be accomplished by adding an embedded layer of soft thermoplastic and/or a layer of acrylic, alloy, composite or a similar material on the casts.

A vacuum forming procedure with or without any cement material may be placed intraorally. The shape of the auxiliaries enables the adding of different components to the dental aligners.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A method for producing dental aligners, the method comprising:
scanning a patient's teeth to produce a 3D printable file;
adjusting the 3D printable file according to a treatment plan;
printing a dental aligner mold based on the adjusted 3D printable file; and producing dental aligners from the dental aligner mold.

2. The method for producing dental aligners of claim 1, wherein adjusting the 3D printable file includes setting different guidelines.

3. The method for producing dental aligners of claim 1, wherein adjusting the 3D printable file includes defining a gum line.

4. The method for producing dental aligners of claim 1, wherein adjusting the 3D printable file includes moving the patient's teeth from a starting position to a final position.

5. The method for producing dental aligners of claim 1, wherein adjusting the 3D printable file includes making an arch length compatible for a total length of the patient's teeth.

6. The method for producing dental aligners of claim 1, wherein adjusting the 3D printable file includes A.I. suggestions for best adjustments.

7. A dental aligner, comprising:
a lower jaw portion;
an upper jaw portion; wherein each of the lower jaw portion and the upper jaw portion has an outer surface and an inner surface; wherein a thickness is formed between the inner surface and the outer surface, the thickness distributed ununiformly at different positions of each of the lower jaw portion and the upper jaw portion; wherein auxiliaries attach to each of the lower jaw portion and the upper jaw portion to align a user's teeth by different means.
